(19) United States
(12) Patent Application Publication
(10) Pub. No.: US 2026/0335038 A1
(43) Pub. Date: Oct. 29, 2026

(54) ATTACHMENT MODULE FOR AN OPHTHALMIC SURGICAL MICROSCOPE, OPHTHALMIC SURGICAL MICROSCOPE

(71) Applicant: CARL ZEISS MEDITEC AG, Jena (DE)

(72) Inventor: Hessam ROODAKI, Munich (DE)

(21) Appl. No.: 19/655,611

(22) Filed: Apr. 22, 2026

(30) Foreign Application Priority Data
Apr. 23, 2025 (DE) 102025115570.4

(51) Int. Cl. A61B 90/25; A61B 3/00; A61B 3/13; A61B 90/50

(52) U.S. Cl. CPC A61B 90/25; A61B 3/0008; A61B 3/132; A61B 90/50; A61B 2560/0214

(57) ABSTRACT

The present disclosure relates to an attachment module for an ophthalmic surgical microscope. The attachment module comprises a carrying structure and a front lens. The carrying structure is designed to hold the front lens relative to the surgical microscope in a position facing an examined patient's eye. A light source is attached to the front lens. The light source is located radially outside an observation channel provided by the front lens.

The present application claims the priority of the German patent application DE 10 2025 115 570.4, filed on Apr. 23, 2025. The content of that German patent application is incorporated by reference in the present application text.

### FIELD OF THE DISCLOSURE

The present disclosure relates to an attachment module for an ophthalmic surgical microscope, and to an ophthalmic surgical microscope.

### BACKGROUND OF THE DISCLOSURE

Ophthalmic surgical microscopes are used by ophthalmologists to allow them to observe the eye on a magnified scale in the course of an examination or treatment of a patient. The surgical microscope is usually positioned and set such that the surgical microscope produces a magnified image of an anterior segment of the patient's eye. Illumination light can be guided onto the front side of the patient's eye in order to ensure that the image generated by the surgical microscope is sufficiently bright. The illumination system providing the illumination light can be integrated into the surgical microscope such that the illumination light is guided onto the patient's eye through the main objective of the surgical microscope.

The combined use of ophthalmic surgical microscopes and an attachment module for displacing the focal position of the surgical microscope is established practice. As a result, different planes in the patient's eye can be observed without changing the position or setting of the surgical microscope. For example, such an attachment module can be used to switch between a first mode, in which the front side of the patient's eye is observed, and a second mode, in which the fundus – i.e. the background – of the eye is observed.

The internal illumination system of a surgical microscope cannot easily provide the illumination light required for the examination of the fundus. The illumination light would have to pass through the optical elements of the attachment module in order to be able to illuminate the fundus. However, this would give rise to reflections off the surfaces of the optical elements, and these reflections would disturb the image visible in the surgical microscope.

It is therefore known practice to use invasive illumination for the illumination of the interior of the patient's eye. An incision can be produced in the wall of the patient's eye for this purpose, and an end of a light guide is introduced into the interior of the patient's eye through this incision. A brightness of the fundus that is sufficient for the surgical microscope can be generated by illumination light emerging from the end of the light guide. This procedure is disadvantageous inasmuch as the patient's eye is injured on account of the introduction of the light guide.

### SUMMARY OF THE DISCLOSURE

The problem addressed by the present disclosure is that of presenting an attachment module for an ophthalmic surgical microscope and an ophthalmic surgical microscope, by means of which the aforementioned disadvantages are reduced. This problem is solved by the features of the independent patent claims. Advantageous embodiments are specified in the dependent claims.

An attachment module according to the present disclosure for an ophthalmic surgical microscope comprises a carrying structure and a front lens. The carrying structure is designed to hold the front lens relative to the surgical microscope in a position facing an examined patient's eye. A light source is located at the front lens. The light source is located radially outside an observation channel provided by the front lens.

The present disclosure proposes locating a light source at the front lens of the attachment module and illuminating the fundus with illumination light emanating from the light source. The illumination light passes through the transparent structures in the anterior segment of the eye and thus reaches the interior of the patient's eye. Provided the illumination light does not directly illuminate the entire fundus, this gives rise to substantially uniform brightness as a result of multiple reflections off the inner side of the wall of the eye. The fundus can be illuminated in a manner suitable for observation through the surgical microscope without the patient's eye being injured and without the illumination light being reflected off the optical elements of the attachment module.

The front lens refers to the last lens of the attachment module in front of the eye. Between the front side of the eye and the front lens of the attachment module, the observation beam path of the surgical microscope therefore does not pass through any further optical elements of the attachment module.

The attachment module may comprise a mount that is used to hold the front lens on the carrying structure of the attachment module. The light source can be attached to the mount.

The front lens may consist of a suitable transparent material, for example glass or a transparent polymer material. The light source may be attached to the front lens. In one embodiment, the light source is attached to a surface of the front lens. The light source can be suitably connected to a surface of the front lens, for example by adhesive bonding, by soldering, by welding, by way of magnetic forces or in a comparable manner.

The light source can be attached to a front side of the front lens facing the examined patient's eye. This enables a direct light path from the light source in the direction of the patient's eye. It is also possible that the light source is attached to a rear side of the front lens facing away from the examined patient's eye. In that case, the light path of the illumination light may pass through the material of the front lens in the direction of the patient's eye.

In a further embodiment, the light source is introduced into the material of the front lens. This enables a design in which the light source does not project beyond the contour spanned by the front lens. For example, a front lens with a light source introduced into the material thereof can be produced by virtue of the material, when in its liquid state, being cast into a mould and the material curing around the light source. For this purpose, the light source can be inserted into the liquid material, or the liquid material can be cast around the light source, which is already in position. Alternatively, the light source can also be inserted into a cutout in the front lens.

The light source might be an active light source, in which the light emitted by the light source is generated within the light source. The active light source can be an electrically operated light source. It is also possible that the light source is a passive light source, in which the emitted light is produced elsewhere. An example of a passive light source is the distal end of a light guide. The primary passive light source can interact with a secondary active light source, in which the light is generated and from where the light is supplied to the passive light source. The light emitted by the secondary active light source can be fed into the proximal end of a light guide should the distal end of a light guide form the passive light source. The secondary active light source can be a constituent part of the attachment module. It is also possible that the secondary active light source is a constituent part of the surgical microscope. The interface between the attachment module and the surgical microscope may comprise a light guide interface. The light guide interface can be designed such that the primary passive light source is automatically separated from the secondary active light source when the attachment module is separated from the surgical microscope. Conversely, the light guide interface can be connected automatically when the attachment module is connected to the surgical microscope. In one embodiment, the attachment module is a passive attachment module without electrical elements. In that case, the attachment module can be used without the need to establish an electrical connection.

The light source may span a ring shape which extends over the circumference of the front lens. The ring may be a closed ring which extends without interruption over the circumference of the front lens. A ring shape with interruptions is also possible such that illuminated regions and non-illuminated regions alternate within the ring. For example, the light source may be formed by one or more light-emitting diodes (LEDs). Other types of light sources are also possible, for example incandescent light sources, gas discharge light sources, OLEDs, halogen light sources and others.

The light source may be a light source with adjustable operating parameters. For example, the adjustable operating parameters may include the brightness and/or the spectral distribution, in particular the colour temperature. Should the light source comprise multiple LEDs, the latter may be controllable individually or in groups. The LEDs may emit light of different wavelengths such that the spectral composition of the illumination light can be influenced by the activation of suitable combinations of LEDs.

The light source can be attached to a power source, from where the electrical energy required to generate the illumination light is supplied to the light source. The power source may be a power source external to the attachment module. The current may be supplied by way of electrical cables which extend out of the attachment module. The attachment module may comprise electrical contacts, by means of which the connection to the power source can be established or disconnected. The electrical contacts may be a constituent part of an electrical plug-in connection, which can be separated or connected by hand.

The electrical contacts may also be designed such that connection is established automatically when the attachment module is connected to the surgical microscope and released automatically when the attachment module is separated from the surgical microscope. The power supply may comprise a portion extending within the body of the surgical microscope. This allows the power supply to be derived from the same power source that also supplies the surgical microscope.

The power source may be a battery. The battery can be a constituent part of the attachment module. This allows the attachment module to be used without the need for connecting and disconnecting electrical contacts when the attachment module is inserted into and removed from the observation beam path, respectively.

The attachment module may comprise an interface with both an electrical connection and a light guide connection. The light guide connection allows the transmission of light, which was emitted by the light source, between a primary active light source and a secondary passive light source. For example, it may be possible to influence parameters such as the direction of the emitted light by way of the electrical connection.

In one embodiment, the attachment module comprises a main component intended for multiple uses and a disposable component intended for a single use. The disposable component can be disposed of after being used in connection with one patient. A new disposable component can be used for the next patient. The front lens can be a constituent part of the disposable component. Additional maintenance outlay as a result of the battery can be avoided if the battery which supplies the light source is also a constituent part of the disposable component. The battery capacity can be dimensioned such that the battery is capable of providing electrical energy for the duration of typical examinations or treatments of the patient. Reliable supply of the light source is always ensured since a new disposable component with a new battery is used for the next patient.

A switch used to switch on the light source may be provided. The switch can be a constituent part of the attachment module. In particular, the switch can be a constituent part of the disposable component of the attachment module. In the case of a disposable component, the attachment module can be designed such that the light source shines until the battery is empty, which is why it is not necessary to be able to switch the light source off again using the switch. In many cases, the switch does also offer the option of switching the light source off again.

In addition to that, or as an alternative, it is possible to provide one or more actuating elements which can be used to change operating parameters of the light source such as the brightness and/or the spectral composition of the illumination light. The actuating element can be a constituent part of the attachment module. The actuating element(s) can also be constituent part(s) of the surgical microscope or a separate structural unit.

Signal transmission between the actuating element and the light source can be effected by way of an electrical conductor path, for example in the form of a cable, an electrical conductor inserted into the material or comparable electrical conductor paths. The electrical conductor path may be attached to the structure of the attachment module or embedded in the structure of the attachment module. A dedicated electrical conductor path may be provided for each of the operating parameters to be set should multiple light sources and/or multiple operating parameters of a light source be controlled. It is also possible that multiple operating parameters are controllable by way of a joint electrical conductor path. For this purpose, control signals can be transmitted using a suitable protocol. For example, a first signal sequence may specify the operating parameter to be adjusted, and a second signal sequence may specify the value to which the operating parameter in question is set.

There can be a less complex electrical interface between the actuating element and the light source if fewer electrical conductor paths are provided for controlling the light source. In one embodiment there are separate conductor paths for the power supply and the control of the operating parameters of the light sources. In the case of a suitable modulation of the electrical signals, the conductor paths used for supplying power can also be used for transmitting control signals. In that case, both the power supply and the control signals can be provided by way of only two poles. For example, there may be separable electrical interfaces between the disposable component and the main component of the attachment module and/or between the attachment module and the surgical microscope.

The attachment module can be designed such that illumination light emitted by the light source can propagate in a straight line in the direction of the patient's eye. The illumination light path may also be deflected one or more times between the light source and the patient's eye. For example, the illumination light emitted by the light source can be guided onto a deflection element before the illumination light is incident on the patient's eye. The deflection element can be designed such that the illumination light is reflected off the deflection element before it is incident on the patient's eye.

An optical filter may be arranged in the beam path of the illumination light between the light source and the patient's eye such that a portion of the illumination light passes through the optical filter, and another portion of the illumination light is reflected off the optical filter. The illumination light can be spectrally split in this way such that the components of the illumination light that contribute to the illumination of the patient's eye in a desired manner are incident on the patient's eye, while other components, which would for example primarily lead to tissue heating, are not guided to the patient's eye. The attachment module can be designed such that components of the illumination light reflected off the optical filter are incident on the patient's eye. Alternatively, the attachment module can be designed such that components of the illumination light passing through the optical filter are incident on the patient's eye.

The direction of the illumination light emitted by the light source may be adjustable. For example, this can be achieved by virtue of controlling different regions or parts of the light source in such a way that the direction of the emitted illumination light changes. It is also possible that the attachment module comprises actuators which are designed to mechanically change the alignment of the light source or of parts of the light source.

The attachment module can be designed such that the front lens is held in its desired position by gravity when the attachment module is used with the surgical microscope. Further, the attachment module can be designed such that the front lens can be deflected upwards when a force acts on the front lens from below. This can avoid injury to the patient's eye should the surgical microscope inadvertently be moved too far downwards. In that case, the front lens can be deflected instead of injuring the patient's eye. For example, a suitable joint can be formed in the carrying structure of the attachment module in order to allow for the deflection movement.

When the attachment module is used on a patient's eye, the front lens can generate an intermediate image in an intermediate image plane arranged between the front lens and the main objective of the surgical microscope. The attachment module may comprise optical elements that are used to shape the observation beam path between the intermediate image plane and the main objective of the surgical microscope. The observation beam path enters the optical system of the surgical microscope via the main objective. In particular, the optical elements of the attachment module can be used to shape the observation beam path such that an image of the fundus is generated in an image plane of the surgical microscope. This is preferably effected without needing to change the optical system in comparison with a state in which the surgical microscope is used to observe the front side of the eye without using the attachment module.

The present disclosure also relates to a system made of a surgical microscope and an attachment module according to the present disclosure. The surgical microscope is intended to define an observation beam path which extends between the patient's eye and a main objective of the surgical microscope. The attachment module is arranged outside the observation beam path in a first system state. The observation beam path passes through the attachment module in a second system state.

### BRIEF DESCRIPTION OF THE DRAWINGS

The present disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 shows a schematic illustration of an ophthalmic surgical microscope, according to aspects of the present disclosure.

FIG. 2 shows a surgical microscope with an attachment module, according to aspects of the present disclosure.

FIG. 3 shows the attachment module from FIG. 2 in an enlarged representation, according to aspects of the present disclosure.

FIG. 4 shows a view of the attachment module from FIG. 3 from below, according to aspects of the present disclosure.

FIG. 5 shows a front lens of an attachment module, according to aspects of the present disclosure.

FIG. 6 shows the view according to FIG. 3 in an alternative embodiment of the present disclosure;

FIG. 7 shows a view of the attachment module from FIG. 6 from below, according to aspects of the present disclosure.

FIG. 8 shows the view according to FIG. 7 in an alternative embodiment of the present disclosure;

FIG. 9 shows an aspect of the embodiment from FIG. 8, according to aspects of the present disclosure.

FIGS. 10-11 shows the view according to FIG. 5 in alternative embodiments of the present disclosure;

FIG. 12 shows the front lens of an attachment module according to the disclosure in an alternative embodiment of the present disclosure;

FIGS. 13-14 shows the view according to FIG. 12 in further embodiment of the present disclosure;

FIG. 15 shows the view according to FIG. 3 in a further embodiment of the present disclosure.

### DETAILED DESCRIPTION

Various embodiments are described with reference to the attached figures, where like reference numerals are used throughout the figures to designate similar or equivalent elements. Each reference numeral identifies the figure in which the reference numeral first appears based on the first digit (for three-digit reference numerals) or the first two digits (for four-digit reference numerals) corresponding to the figure numeral of the figure. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

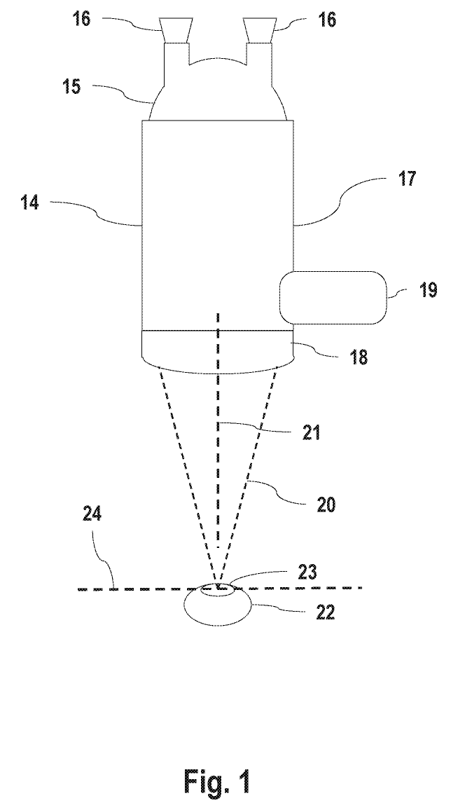

An ophthalmic surgical microscope 14 shown in FIG. 1 defines a stereoscopic observation beam path 20, which extends from an object in the form of a patient's eye 22 via a main objective 18 and via a magnification system arranged in a microscope body 17 to a binocular head 15 with two eyepieces 16. An observer directing their gaze through the two eyepieces 16 sees a magnified image from the object plane 24, which is located in the anterior segment 23 of the patient's eye 22 in FIG. 1.

The surgical microscope 14 is equipped with an illumination system 19 which emits illumination light. The illumination light passes through the main objective 18 and illuminates the object plane. The image visible in the eyepieces 16 has sufficient brightness on account of the illumination light.

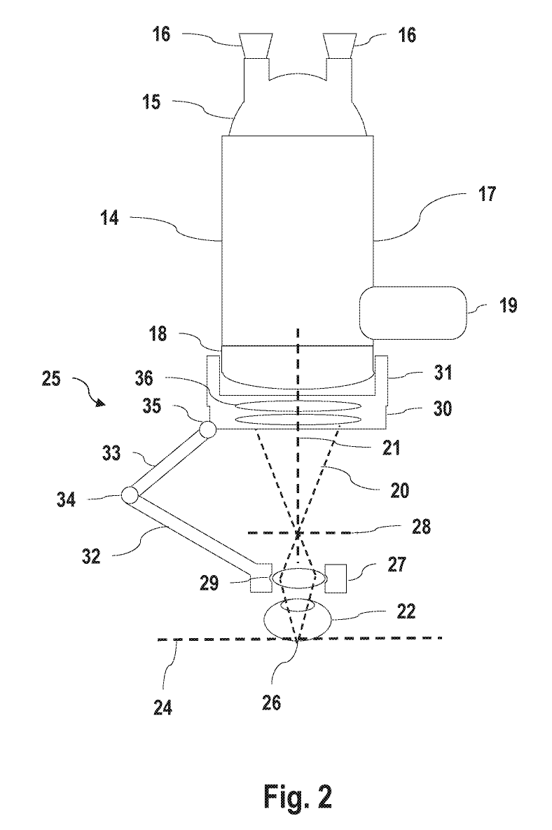

FIG. 2 depicts the surgical microscope 14 from FIG. 1 in a different state, in which an attachment module 25 is arranged between the main objective 18 and the patient's eye 22. The attachment module 25 comprises a housing 30 which is connected to the surgical microscope 14 by way of a connecting mechanism 31. The housing 30 accommodates multiple lenses 37 which modify the observation beam path 20 together with a front lens 29 of the attachment module 25 such that the object plane is located in the posterior segment 26 of the patient's eye 22. This yields an observation beam path 20 which extends from the posterior segment 26 of the eye through the transparent structures in the anterior segment 23 of the eye and the front lens 29 and via an intermediate image plane 28 and the further lenses 37 of the attachment module 25 into the surgical microscope 14. The image inversion occurring with the intermediate image plane 28 is inverted again in the further course such that the observer sees an upright image of the fundus 26 in the eyepieces 16.

The front lens 29 held in a mount 27 is held by the housing 30 of the attachment module 25 by way of a first strut 32 and a second strut 33. A first pivot joint 34 is formed between the first strut 32 and the second strut 33. A second pivot joint 35 is formed between the second strut 33 and the housing 30 of the attachment module 25. Under the influence of gravity, the front lens 29 is held in the position shown in FIG. 2 by way of the struts 32, 33 and the pivot joints 34, 35. Under the influence of an upwardly acting force, the mount 27 with the front lens 29 can pivot upward, and so the front lens 29 cannot exert higher pressures on the patient's eye 22.

In FIG. 2, the illumination system 19 of the surgical microscope 14 cannot be used to introduce illumination light into the object plane 24 because illumination light emerging from the main objective 18 would cause undesired reflections off the optical elements of the attachment module 25, i.e. off the lenses 37 and the front lens 29. According to the present disclosure, the attachment module 25 is therefore equipped with its own light source 36 for illumination light. The light source 36 is arranged such that the illumination light can propagate directly in the direction of the patient's eye 22 without impinging on the lenses 29, 37 of the attachment module 25.

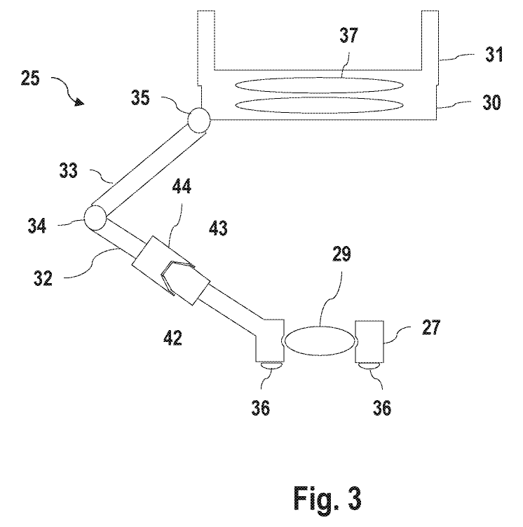

In the enlarged illustration of the attachment module 25 in FIG. 3, the light source 36 is attached to the underside of the mount 27 holding the front lens 29. The mount 27 extends in ring-shaped fashion around the front lens 29. The light source 36 also spans a ring shape over the circumference of the front lens 29. Illumination light emanating from the light source 36 can propagate unimpeded in the direction of the patient's eye 22 and enter the interior of the patient's eye 22 through the transparent structures in the anterior segment 33 of the eye. There the illumination light ensures sufficient brightness of the fundus 26, optionally after one or more reflections off the inner wall of the patient's eye. A first part of the attachment module 25 can be separated from a second part of the attachment module 25 by way of a coupling 42.

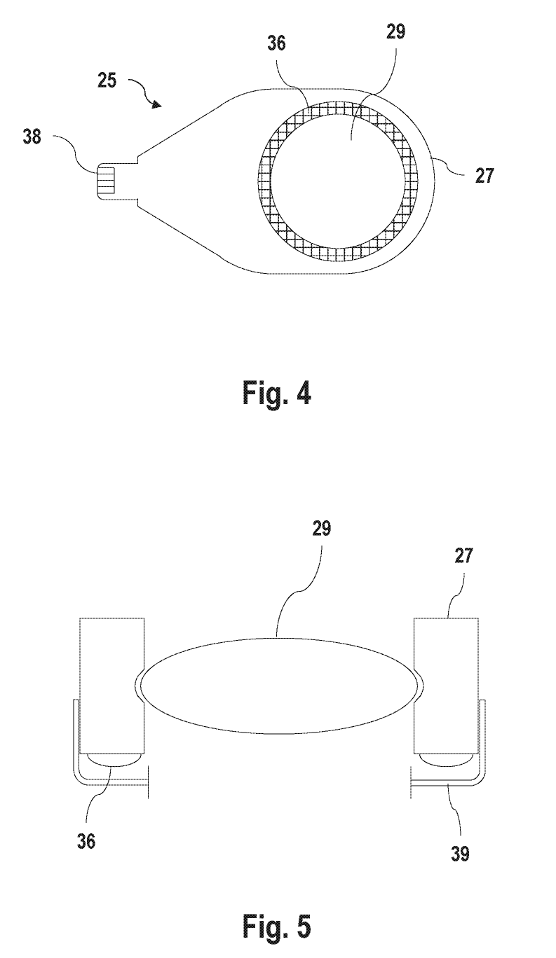

FIG. 4 shows a view of the attachment module 25 from FIG. 3 from below. Accordingly, the light source 36 extends as a light band in the form of a closed ring around the front lens 29. The light source 36 is located radially outside the observation channel formed by the front lens 29, and so the view of the user directing their gaze through the eyepieces 16 of the surgical microscope 14 is not impaired.

The attachment module 25 is equipped with an electrical interface 38 which comprises four electrical contacts. When the attachment module 25 is connected to the surgical microscope 14, a cable coming from an operating unit is connected to the electrical interface 38 by means of a plug. The light source 36 is supplied with electrical energy by means of two of the four contacts. Control commands allowing the brightness of the light source 36 to be set can be transmitted to the light source 36 by means of the two other contacts.

In the alternative embodiment according to FIG. 5, the attachment module 25 comprises an optical filter 39 connected to the mount 27. Illumination light emitted by the light source 36 passes through the optical filter 36 on its way to the patient's eye 22. The optical filter 36 is configured such that long-wavelength light with a wavelength above a threshold value is reflected, while short-wavelength light with a wavelength below the threshold value can pass. The threshold value is chosen such that the components of the emitted illumination light which primarily lead to tissue heating and contribute little to the brightness are not incident on the patient's eye 22.

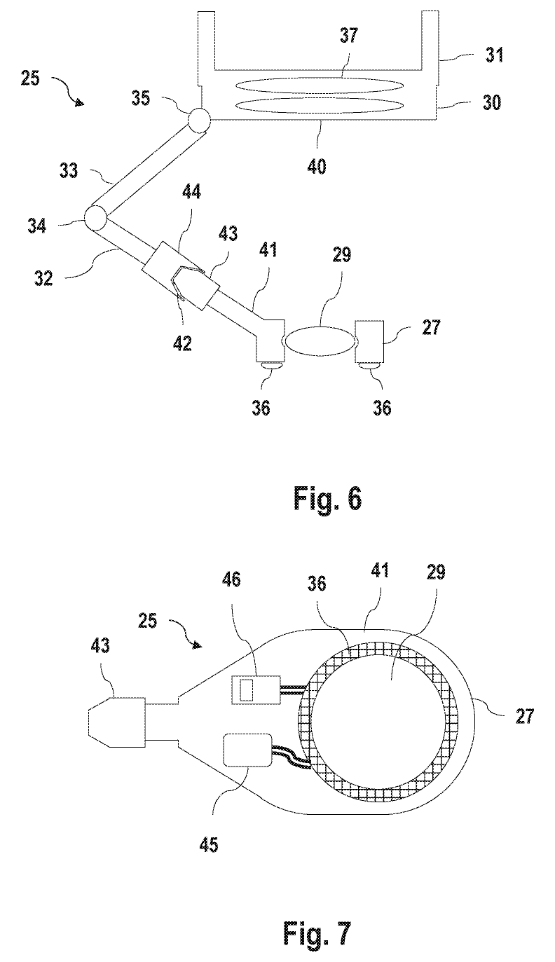

FIGS. 6, 7 show an alternative embodiment in which the attachment module 25 is composed of a main component 40 and a disposable component 41. The main component 40 is intended for multiple uses. The disposable component 41 is used in connection with one patient and then disposed of. A coupling 42 is formed within the first strut 32 of the attachment module 25. The coupling 42 is composed of a first coupling part 43, which is connected to the disposable component 41, and a second coupling part 44, which is connected to the main component 40. The coupling 42 is separated in order to dispose of the disposable component 41.

According to the schematic illustration in FIG. 7, a disposable component 41 is equipped with a battery 45 and a switch 46. The light source 36 can be switched on and off by way of the switch 46. The capacity of the battery 45 is dimensioned such that the light source 36 can be operated for the expected duration of treatment of a single patient.

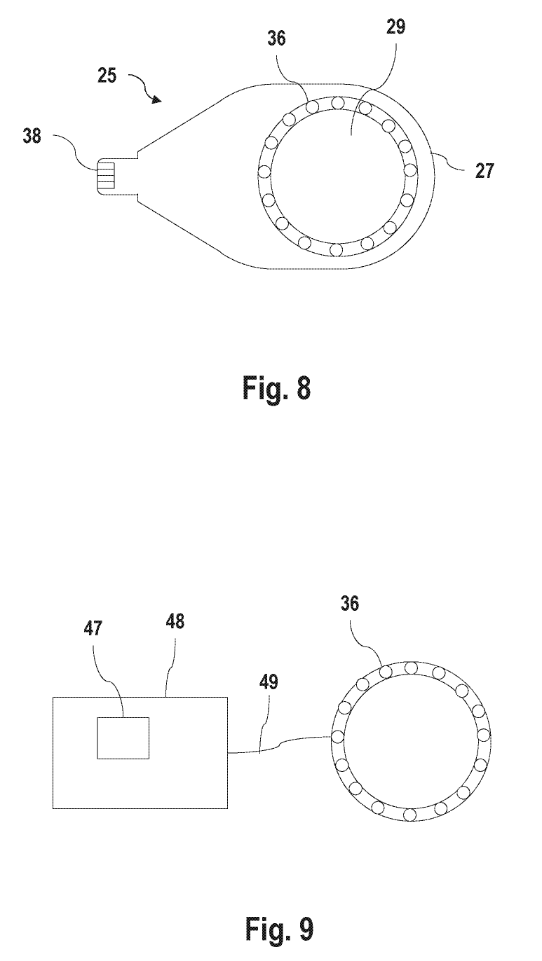

In the alternative embodiment according to FIGS. 8, 9, the light source 36 comprises a plurality of LEDs which are distributed in ring-shaped fashion over the circumference of the front lens 29. The LEDs can be switched on and off on an individual basis. The LEDs emit light with different frequency distribution such that the spectral composition of the illumination light can be changed by suitably controlling the LEDs. Control is implemented by way of a control unit 48 of the surgical microscope 14, which is equipped with an operating unit 47. There is an electrical connection 49 with an interface (not shown) between the control unit 48 and the light source 36. The interface is connected automatically when the attachment module 25 is connected to the surgical microscope 14 and disconnected automatically when the attachment module 25 is disconnected from the surgical microscope 14. In an addition to the electrical connection, or as an alternative, the interface may comprise a light guide connection which can be disconnected at the interface. The light guide connection can be used to supply light to a light source 36 in the form of a passive light source. The light guide and/or the electrical conductor paths may be attached to the structure of the attachment module or embedded in the structure of the attachment module.

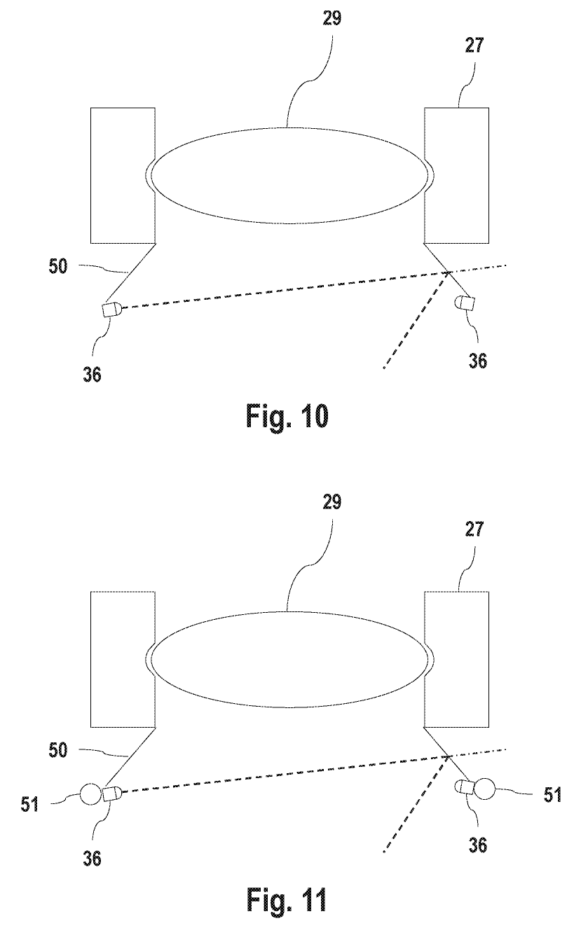

FIG. 10 shows a further embodiment in which an optical filter 50 extends downwards in conical fashion from the mount 27. The LEDs of the light source 36 are aligned such that the emitted illumination light is incident on the optical filter 50. Long-wavelength components of the light pass through the optical filter 50; short-wavelength components are reflected. The limit wavelength is chosen such that long-wavelength radiation, which primarily heats the tissue, is not guided onto the patient's eye 22. In the variant according to FIG. 11, the LEDs of the light source 36 are connected to one another by means of a ring 51 with an adjustable diameter. Adjusting the ring 51 changes the position of the LEDs and of the optical filter 55 such that the light is incident on the patient's eye 22 at a different angle. This can be used to influence the light distribution in the posterior segment of the eye in a desired manner.

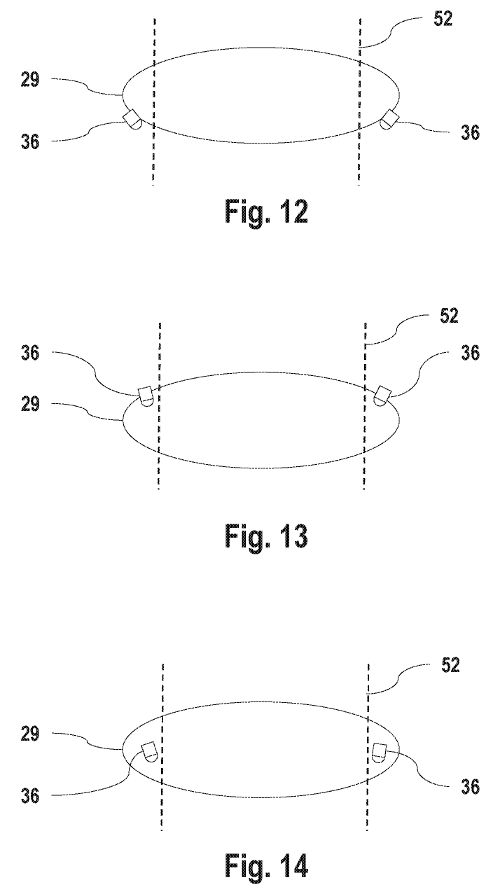

FIG. 12 shows a variant in which the LEDs of the light source 36 are attached directly to the underside of the front lens 29. The LEDs are arranged in a peripheral position on the front lens 29 such that the observation channel 52, which is provided by the front lens 29 and within which the observation beam path 20 of the surgical microscope 14 extends, is not impaired. In FIG. 13, the LEDs of the light source 36 are attached to the top side of the front lens 29. The illumination light emitted by the LEDs propagates through the material of the front lens 29 in the direction of the patient's eye 22. In the variant according to FIG. 14, the LEDs of the light source 36 are located within the front lens 29, i.e. are surrounded all around by the material of the front lens 29. The front lens 29 consists of a transparent polymer material which, in a liquid state, is cast into a mould in which the LEDs 36 have already been positioned. The LEDs 36 are securely embedded in the material of the front lens 29 after the polymer material has cured. The electrical connections (not shown in FIG. 14) are guided out of the front lens 29 to the outside.

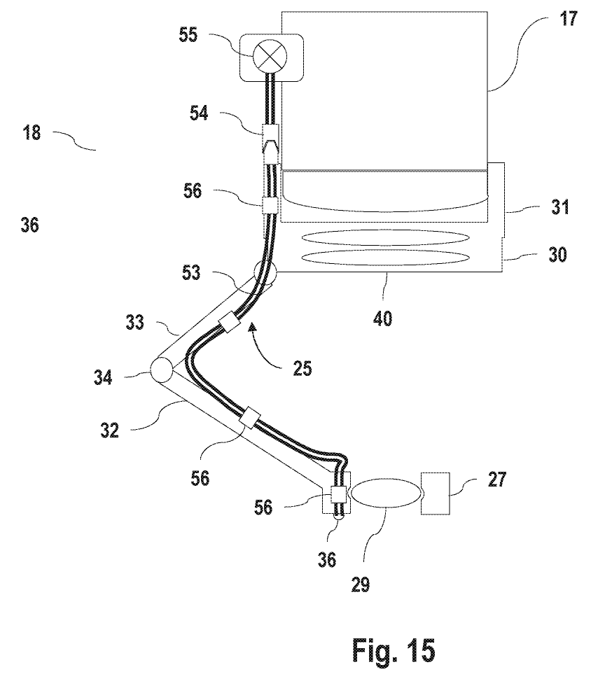

FIG. 15 shows an embodiment in which the light source 36 is formed by the distal end of a light guide 53. The light source 36 thus is a primary passive light source, which interacts with a secondary active light source 55. The secondary active light source 55 feeds the emitted light into the proximal end of the light guide 53 and thus provides the light emitted by the primary passive light source 36.

The secondary active light source 55 can be integrated into the housing of the surgical microscope 14. The light guide 53 may contain a light guide interface 54 which is automatically connected when the attachment module is connected to the surgical microscope and which is automatically disconnected when the attachment module 25 is disconnected from the surgical microscope. Holding elements 56 may be used to attach the light guide 53 to the structure of the attachment module 25. In this exemplary embodiment, the attachment module 25 is a passive attachment module without electrical components.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of the claims below can be combined with one or more elements or aspects or steps, or any portion(s) thereof, from one or more of any of the other claims below or combinations thereof, to form one or more additional implementations and/or claims of the present disclosure.

What is claimed is:

1. An attachment module for an ophthalmic surgical microscope, the attachment module comprising:
a light source;
a carrying structure; and
a front lens,
wherein the carrying structure is designed to hold the front lens relative to the surgical microscope in a position facing an examined patient's eye,
wherein the light source is located at the front lens, and
wherein the light source is located radially outside an observation channel provided by the front lens.

2. The attachment module according to claim 1, wherein the front lens is held in a mount and wherein the light source is attached to the mount.

3. The attachment module according to claim 1, wherein the light source is attached to the front lens

4. The attachment module according to claim 3, wherein the light source is embedded in the material of the front lens.

5. The attachment module according to any of claim 1, wherein the light source is a primary passive light source which interacts with a secondary active light source spaced apart from the attachment module.

6. The attachment module according to any of claim 1, wherein the light source spans a ring shape which extends over a circumference of the front lens.

7. The attachment module according to any of claim 1, further comprising electrical contacts for supplying power to the light source, wherein the electrical contacts are designed to automatically establish electrical contact with mating contacts of the surgical microscope when the attachment module is connected to the ophthalmic surgical microscope.

8. The attachment module according to any of claim 1, further comprising a main component configured for multiple uses and a disposable component configured for a single use, wherein the disposable component comprises the front lens.

9. The attachment module according to claim 8, wherein the disposable component is equipped with a battery for supplying power to the light source.

10. The attachment module according to any of claim 1, wherein an optical filter is arranged in a beam path of a illumination light between the light source and the patient's eye.

11. The attachment module according to claim 10, wherein the illumination light guided onto the patient's eye is reflected off the optical filter.

12. The attachment module according to any of claim 1, further comprising an actuator designed to change the direction of an illumination light emitted by the light source.

13. A system comprising:
a surgical microscope including a main objective; and
an attachment module including:
a light source;
a carrying structure; and
a front lens, wherein the carrying structure is designed to hold the front lens relative to the surgical microscope in a position facing an examined patient's eye, wherein the light source is located at the front lens, and wherein the light source is located radially outside an observation channel provided by the front lens;
wherein the surgical microscope is designed to define an observation beam path which extends between a patient's eye and the main objective of the surgical microscope,
wherein the attachment module is arranged outside the observation beam path in a first system state, and
wherein the observation beam path passes through the attachment module in a second system state.
